(19) United States
(12) Patent Application Publication
(10) Pub. No.: US 2023/0409898 A1
(43) Pub. Date: Dec. 21, 2023

(54) PROTEIN STRUCTURE PREDICTION USING MACHINE LEARNING

(71) Applicants: International Business Machines Corporation, Armonk, NY (US); MIT - Massachusetts Institute of Technology, Cambridge, MA (US)

(72) Inventors: Pin-Yu Chen, White Plains, NY (US); Siyu Huo, White Plains, NY (US); Tengfei Ma, White Plains, NY (US); Lingfei Wu, Elmsford, NY (US); Kai Guo, Singapore (SG); Federica Rigoldi, Milan (IT); Benedetto Marelli, Lexington, MA (US); Markus Jochen Buehler, Boxford, MA (US)

(21) Appl. No.: 17/842,839

(22) Filed: Jun. 17, 2022

(51) Int. Cl. G06N 3/08; G06N 3/04

(52) U.S. Cl. CPC G06N 3/08; G06N 3/04

(57) ABSTRACT

A system may include a memory and a processor in communication with the memory. The processor may be configured to perform operations. The operations may include training a neural network and predicting structural feature sets with the neural network. The operations may include producing predicted structures with the neural network using the structural feature sets, converting the predicted structures into predicted graphs with predicted edges, and comparing predicted graphs to training graphs and predicted edges to training edges to obtain a comparison. The operations may include training a model with the comparison, constructing a graph with the neural network using a node feature set, and reducing missing edges in the graph with the model.

### BACKGROUND

The present disclosure relates to the field of materials science, and more specifically to artificial intelligence in bioengineering, medicine, and materials science applications.

Molecular prediction, discovery, design, synthesis, and testing often takes a substantial amount of time, data, and calibration. The process may use various computational tools. Such tools be used to predict, design, discover, test, and/or synthesize molecules and/or molecular structures. Computational tools may include neural networks, including deep neural networks, and graph neural networks. Prediction computational tools may be used predict molecular structures, including the structures of peptides, polypeptides, and proteins.

Molecular structure predictions advance scientific discovery. Molecular structure predictions based on input sequences may be increasingly less accurate as molecular structure complexity increases. Predicting beta strands can result in accuracy issues, and current mechanisms provide no means to correct initial predictions.

### SUMMARY

Embodiments of the present disclosure include a system, method, and computer program product for molecular design, discovery, prediction, and synthesis. Embodiments of the present disclosure may enable improved accuracy for predicting structures based on input sequences, including improving initial predictions. Embodiments of the present disclosure may leverage features, chemical data, biological data, and/or domain knowledge for structural predictions. Some embodiments may include leveraging known sequence features and measurements to improve predictions, and some features may be used as regularizers.

A system in accordance with the present disclosure may include a memory and a processor in communication with the memory. The processor may be configured to perform operations. The operations may include training a neural network (NN) and predicting structural feature sets with the neural network. The operations may include producing predicted structures with the neural network using the structural feature sets, converting the predicted structures into predicted graphs with predicted edges, and comparing predicted graphs to training graphs and predicted edges to training edges to obtain a comparison. The operations may include training a model with the comparison, constructing a graph with the neural network using a node feature set, and reducing missing edges in the graph with the model.

In some embodiments of the present disclosure, the neural network may be a multi-scale neighborhood-based neural network (MNNN). In some embodiments, the multi-scale neighborhood-based neural network may be a multi-goal multi-scale neighborhood-based neural network (multi-goal MNNN).

In some embodiments of the present disclosure, the model may be a variational graph auto-encoder (VGAE).

In some embodiments of the present disclosure, the test missing edges may be reduced below a threshold value.

In some embodiments of the present disclosure, the operations may include inputting amino acid code into the neural network with the structural feature set, and the amino acid code may be used to produce the predicted structures.

In some embodiments of the present disclosure, the operations may include using a molecular simulation program to produce the predicted structures.

In some embodiments of the present disclosure, the operations may include labeling differences between predicted graphs and training graphs and labeling differences between predicted edges and training edges.

In some embodiments of the present disclosure, the operations may include predicting a plurality of features of the predicted structure selected from the group comprising dihedral angles, B-factor, solvent-accessible surface area, long-range angles, and short-range angles.

A computer-implemented method in accordance with the present disclosure may include training a neural network and predicting structural feature sets with the neural network. The method may include producing predicted structures with the neural network using the structural feature sets, converting the predicted structures into predicted graphs with predicted edges, and comparing predicted graphs to training graphs and predicted edges to training edges to obtain a comparison. The method may include training a model with the comparison, constructing a graph with the neural network using a node feature set, and reducing missing edges in the graph with the model.

In some embodiments of the present disclosure, the neural network may be a multi-scale neighborhood-based neural network (MNNN). In some embodiments, the multi-scale neighborhood-based neural network may be a multi-goal multi-scale neighborhood-based neural network (multi-goal MNNN).

In some embodiments of the present disclosure, the model may be a variational graph auto-encoder (VGAE).

In some embodiments of the present disclosure, the test missing edges may be reduced below a threshold value.

In some embodiments of the present disclosure, the method may include inputting amino acid code into the neural network with the structural feature set, and the amino acid code may be used to produce the predicted structures.

In some embodiments of the present disclosure, the method may include using a molecular simulation program to produce the predicted structures.

In some embodiments of the present disclosure, the method may include labeling differences between predicted graphs and training graphs and labeling differences between predicted edges and training edges.

In some embodiments of the present disclosure, the method may include predicting a plurality of features of the predicted structure selected from the group comprising dihedral angles, B-factor, solvent-accessible surface area, long-range angles, and short-range angles.

A computer program product in accordance with the present disclosure may include a computer readable storage medium having program instructions embodied therewith. The program instructions may be executable by a processor to cause the processor to perform a function. The function may include training a neural network and predicting structural feature sets with the neural network. The function may include producing predicted structures with the neural network using the structural feature sets, converting the predicted structures into predicted graphs with predicted edges, and comparing predicted graphs to training graphs and predicted edges to training edges to obtain a comparison. The function may include training a model with the comparison, constructing a graph with the neural network using a node feature set, and reducing missing edges in the graph with the model.

In some embodiments of the present disclosure, the neural network may be a multi-scale neighborhood-based neural network (MNNN). In some embodiments, the multi-scale neighborhood-based neural network may be a multi-goal multi-scale neighborhood-based neural network (multi-goal MNNN).

In some embodiments of the present disclosure, the model may be a variational graph auto-encoder (VGAE).

In some embodiments of the present disclosure, the test missing edges may be reduced below a threshold value.

In some embodiments of the present disclosure, the function may include inputting amino acid code into the neural network with the structural feature set, and the amino acid code may be used to produce the predicted structures.

In some embodiments of the present disclosure, the function may include using a molecular simulation program to produce the predicted structures.

In some embodiments of the present disclosure, the function may include labeling differences between predicted graphs and training graphs and labeling differences between predicted edges and training edges.

In some embodiments of the present disclosure, the function may include predicting a plurality of features of the predicted structure selected from the group comprising dihedral angles, B-factor, solvent-accessible surface area, long-range angles, and short-range angles.

The above summary is not intended to describe each illustrated embodiment or every implement of the disclosure.

### BRIEF DESCRIPTION OF THE DRAWINGS

The drawings included in the present application are incorporated into, and form part of, the specification. They illustrate embodiments of the present disclosure and, along with the description, serve to explain the principles of the disclosure. The drawings are only illustrative of certain embodiments and do not limit the disclosure.

FIG. 1 illustrates a system workflow in accordance with some embodiments of the present disclosure.

FIG. 2 depicts a contact map prediction correction mechanism in accordance with some embodiments of the present disclosure.

FIG. 3 illustrates a comparison graph set in accordance with some embodiments of the present disclosure.

FIG. 4 depicts a validation average precision score graph in accordance with some embodiments of the present disclosure.

FIG. 5 illustrates a learning curve graph in accordance with some embodiments of the present disclosure.

FIG. 6 depicts a structure prediction method in accordance with some embodiments of the present disclosure.

FIG. 7 illustrates a cloud computing environment, in accordance with embodiments of the present disclosure.

FIG. 8 depicts abstraction model layers, in accordance with embodiments of the present disclosure.

FIG. 9 illustrates a high-level block diagram of an example computer system that may be used in implementing one or more of the methods, tools, and modules, and any related functions, described herein, in accordance with embodiments of the present disclosure.

While the invention is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the invention.

### DETAILED DESCRIPTION

Aspects of the present disclosure relate to the field of materials science, and more specifically to AI in bioengineering, medicine, and materials science applications.

A multi-scale neighborhood-based neural network (MNNN) may be used in an end-to-end approach to predict the folded structures of molecules such as α-helical proteins. The MNNN may be made applicable to the structural prediction of β-sheets using a machine learning (ML) framework using on a graph neural network (GNN) to correct the MNNN prediction of β-sheet structures. Corrections may be made by identifying the contacts between amino acids predicted by the MNNN that could be improved.

Predicted protein structures from the MNNN and selected features may be provided as input to a GNN. Features may include, for example, dihedral angles, B-factor, solvent-accessible surface area (SASA), long-range angles between amino acids, and short-range angles between amino acids; a multi-goal MNNN model may be built to predict these features. The ML framework for the initial prediction from the MNNN and the correction based on the GNN may be used to improve predictions of molecular structure such as protein structure and protein folding structure.

An MNNN model may be used for predicting dihedral angles of α-helical proteins from amino acid sequences. To learn how amino acid sequences fold into the motifs other than α-helices (e.g., β-sheet folding), even a relatively high prediction accuracy of local structural features such as dihedral angles may not be sufficient because errors on specific dihedral angles may lead to significant discrepancy between the ground-truth and predicted protein structures. A framework to leverage the structures predicted from the existing MNNN models may be used to overcome this limitation. The framework may also extract and use the features representing long-range interactions and/or global features of proteins to correct the predicted structures from the MNNN.

An MNNN model may be trained for predicting dihedral angles. The MNNN model may be further trained into a multi-goal MNNN to enable the prediction of several selected features. Such features may include, for example, B-factor (which may be referred to as the Debye-Waller factor), SASA, dihedral angles (φ and ψ), long-range angles between amino acids, and short-range angles between amino acids. A list of node features may be found in Table 1.

TABLE 1
List of Node Features

| Feature name | Encoding | Description |
|---|---|---|
| amino acid | one-hot | each node represents an amino acid |
| | | residue (one letter code) |
| φ − ψ angles | one-hot | index of the cluster the φ − ψ angle |
| | | combination belongs to |
| B-factor | value | averaged B-factor of the residue |
| SASA | value | solvent-accessible surface area |
| long-range angle | value | Cα(i) − Cα(i + N) − Cα(i + 2N), N = 3 |
| short-range angle | value | Cα(i) − Cα(i + 1) − Cα(i + 3) |

Such features may be or include structural features. One or more of such features may be available from a protein data bank (PDB). Contact maps of molecular structures predicted by the MNNN and one or more of these features may be used to construct molecular graphs. In some embodiments of the present disclosure, the features may be protein structural features, and the protein structural features may be used to construct protein graphs.

The molecular graphs may be input into a variational graph auto-encoder (VGAE) model. The VGAE model may output ground-truth protein structure contacts between amino acids that are not predicted by the MNNN. The VGAE outputs may be leveraged to correct the protein structures predicted by the MNNN to achieve a higher accuracy on the prediction of protein structures, particularly β-sheet structures and other motifs. Such an approach may correct MNNN model predictions and suggest additional strategies for further improving folding approaches.

In some embodiments of the present disclosure, the approach may use GNNs and utilize graphical representations of proteins. In some embodiments, the approach may correct protein structure predictions from an MNNN model such as a multi-goal MNNN model. In some embodiments, the approach may output one or more suggestions for new strategies to further improve existing protein folding strategies.

A system in accordance with the present disclosure may include a memory and a processor in communication with the memory. The processor may be configured to perform operations. The operations may include training a neural network and predicting structural feature sets with the neural network. The operations may include producing predicted structures with the neural network using the structural feature sets, converting the predicted structures into predicted graphs with predicted edges, and comparing predicted graphs to training graphs and predicted edges to training edges to obtain a comparison. The operations may include training a model with the comparison, constructing a graph with the neural network using a node feature set, and reducing missing edges in the graph with the model.

In some embodiments of the present disclosure, the neural network may be a multi-scale neighborhood-based neural network (MNNN). In some embodiments, the multi-scale neighborhood-based neural network may be a multi-goal multi-scale neighborhood-based neural network (multi-goal MNNN).

In some embodiments of the present disclosure, the model may be a variational graph auto-encoder (VGAE).

In some embodiments of the present disclosure, the test missing edges may be reduced below a threshold value.

In some embodiments of the present disclosure, the operations may include inputting amino acid code into the neural network with the structural feature set; the amino acid code may be used to produce the predicted structures.

In some embodiments of the present disclosure, the operations may include using a molecular simulation program to produce the predicted structures.

In some embodiments of the present disclosure, the operations may include labeling differences between predicted graphs and training graphs and labeling differences between predicted edges and training edges.

In some embodiments of the present disclosure, the operations may include predicting a plurality of features of the predicted structure selected from the group comprising dihedral angles, B-factor, solvent-accessible surface area, long-range angles, and short-range angles.

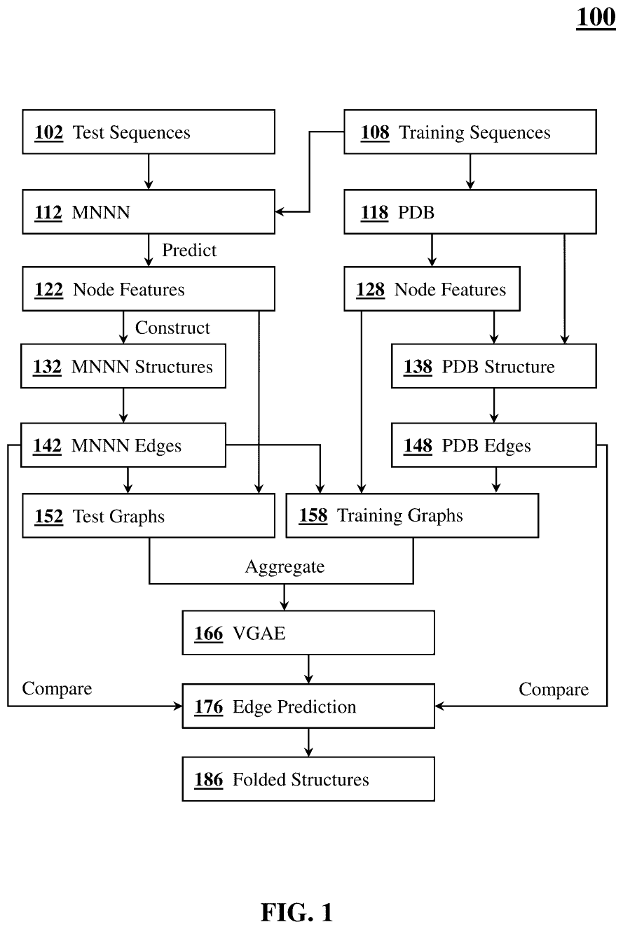

FIG. 1 illustrates a system workflow 100 in accordance with some embodiments of the present disclosure. The workflow 100 uses a VGAE 166 to improve the accuracy of a molecular structure prediction made by an MNNN 112. The system workflow 100 uses sequences to generate node features 122 and 128, structures, graphs, and folded structures 186. Molecular sequences may be divided into test sequences 102 and training sequences 108 to construct input sequences to input into the MNNN 112 and molecular graphs to input into the VGAE 166.

In some embodiments, molecular structures predicted may be, for example, protein structures; protein sequences with 200 residues or less may be divided into training sequences 108 and test sequences 102. In a protein graph, each node may represent an amino acid residue with selected features defined as node features. Two nodes may be connected by an edge if the distance between the Cα atoms of the residues that those two nodes represent is less than threshold value d. The adjacency matrix of a graph may be equivalent to the contact map of the corresponding protein. The amino acid sequences may be input into a multi-goal MNNN model to obtain the predicted dihedral angles, B-factor, SASA, long-range angles, and short-range angles of each residue in the sequence. These residue-wise properties may serve as node features.

Training sequences 108 may be submitted to an MNNN 112 and a PDB 118. The MNNN 112 and PDB 118 may use the training sequences 108 to render node features 122 and 128 to construct MNNN structures 132 and PDB structures 138, respectively. The MNNN structures 132 may be used to identify MNNN edges 142 and the PDB structures 138 may be used to identify PDB edges 148. The MNNN edges 142 and PDB edges 148 may be compared to identify any edges the MNNN 112 did not accurately predict; edges the MNNN 112 does not accurately predict may be referred to as missing edges. In some embodiments, missing edges may be identified and/or labeled. MNNN edges 142 and PDB edges 148 may be used to render training graphs 158. The training graphs 158 may be submitted to a VGAE 166 to generate an edge prediction 176 and render one or more folded structures 186.

In some embodiments, engaging the workflow 100 with the training sequences 108 may be considered as training the MNNN 112 and/or the VGAE 166. The workflow 100 may be used once with a single set of training sequences 108, once with multiple sets of training sequences 108, multiple times with a single set of training sequences 108, or multiple times with multiple sets of training sequences 108 to train the MNNN 112 and/or the VGAE 166.

A trained MNNN 112 and a trained VGAE 166 may be used to predict molecular structures, e.g., of unknown molecules including molecules with one or more known properties but with unknown structures. Unknown molecules may be submitted to the workflow 100 as test sequences 102. In the workflow 100, the test sequences 102 may be submitted to a trained MNNN 112 to predict node features 122 of the test sequences 102 and use these node features 122 to construct MNNN structures 132 and identify MNNN edges 142. The MNNN structures 132 and MNNN edges 142 may be used to generate test graphs 152 for the test sequences 102. The test graphs 152 may be submitted to the trained VGAE 166 to identify missing edges in the test graphs 152. This identification may be based on the edges the MNNN 112 was identified as being unlikely to predict in the training graphs 158 for the training sequences 108. The predicted missing edges may be used by the VGAE 166 to generate an edge prediction 176 and render folded structures 186 for the test sequences 102 that include corrections for the predicted missing edges.

In some embodiments, the MNNN 112 may be a multi-goal MNNN. A multi-goal MNNN may be derived from an MNNN trained to predict dihedral angles: instead of labeling each sequence with a pair of dihedral angles, training sets may be generated in which each input sequence is labeled with one or more features. Such features may include, for example, B-factor, SASA, long-range angles between amino acids, and short-range angles between amino acids of each residue in the sequence. In some embodiments, the MNNN may label dihedral angles as well as one or more features. The training sets may be input into the MNNN 112 (e.g., as, or as part of, the training sequences 108 input) with random weights, and the architecture of the MNNN 112 may be modified to fit into the size of the label in the dataset.

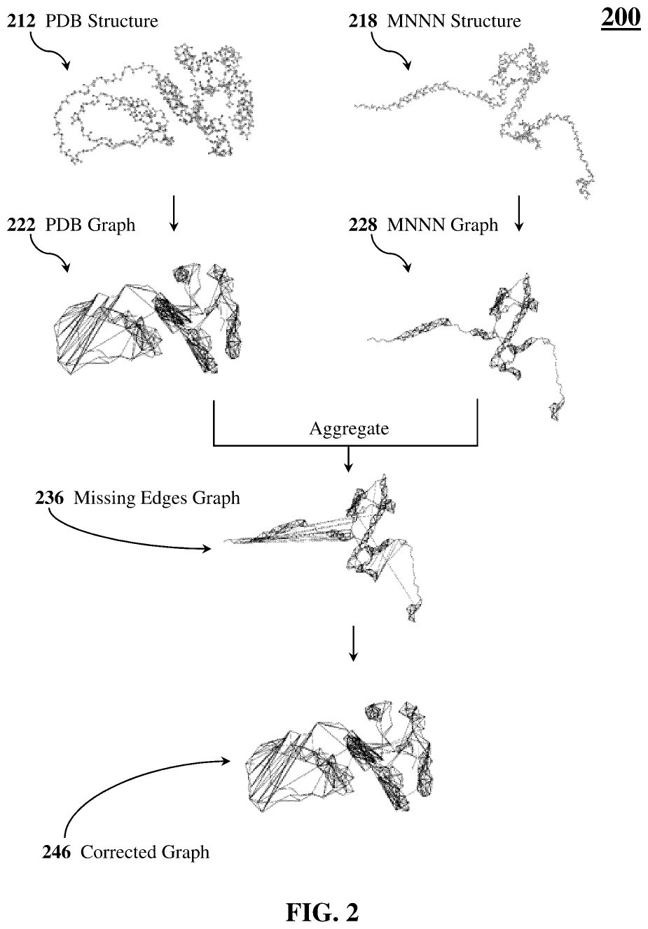

The MNNN 112 may predict node features 122. The node features 122 may include, for example, the amino acid code, B -factor, SASA, dihedral angles (φ and/or ψ), long-range angles between amino acids, short-range angles between amino acids of each residue in the sequence, and the like. The node features 122 may be used to construct MNNN structures 132. The MNNN structures 132 may be molecular structures, peptide structures (e.g., MNNN structure 218 of FIG. 2), protein structures, or similar.

In some embodiments of the present disclosure, an end-to-end model may be developed to render a protein structure prediction starting from a primary amino acid sequence. In such an embodiment, the test input data may be primary sequences, the MNNN 112 may be a multi-goal MNNN, and the test graphs 152 submitted to the VGAE 166 may be generated based on the multi-goal MNNN output.

In some embodiments, test sequences 102 may be fed as input to the multi-goal MNNN to predict features such as the dihedral angles, B-factor, SASA, long-range angles, and short-range angles. The predicted dihedral angles and the amino acid code may be used to reconstruct the MNNN structure 132 which may be a three-dimensional (3-D) all-atom structure. The MNNN structure 132 may be reconstructed by using the script and potential of a molecular simulation program (e.g., the Chemistry at Harvard Macromolecular Mechanics program, also known as CHARMM). The MNNN structure 132 may be converted into a protein graph using any mechanism known in the art or hereinafter developed such as, for example, the mechanisms used for generating PDB protein graphs for training sequences 108.

In some embodiments, training sequences 108 may be input into the workflow 100 for the test sequences 102 to obtain an MNNN 112 prediction of their edges. For training sequences 108, the edges in the protein graphs may be PDB edges 148 constructed from a PDB structure 138 and/or MNNN edges 142 constructed from an MNNN structure 132. The PDB edges 148 and the MNNN edges 142 may be compared to identify any PDB edges 148 not found in the MNNN edges 142 prediction.

The MNNN edges 142 of the training sequences 108 may be combined with the node features 122 extracted by the MNNN 112 from the training sequences 108 to construct the training graphs 158. The MNNN edges 142 and the PDB edges 148 may be compared, and edges found in the PDB edges 148 but not in the MNNN edges 142 may be labeled in the training graphs 158. The training graphs 158 may be input into the VGAE 166 to predict which edges are unlikely to be identified by the MNNN 112 when it predicts the MNNN edges 142 for the test graphs 152. Identifying which edges are unlikely to be identified by the MNNN 112 may enable the system to predict missed edges and compensate therefor. Thus, the edge prediction 176 rendered by the VGAE 166 may be used to improve predictions and render folded structures 186.

Once the MNNN 112 and the VGAE 166 are trained, new sequences may be tested by constructing test graphs 152 using the MNNN edges 142 and node features 122 of the new test sequences 102. One or more corrected protein structures may be obtained by reducing the missing edges in the MNNN edges 142 prediction (as identified in the comparison between the training sequences 108 MNNN edges 142 and the training sequences 108 PDB edges 148). In some embodiments, the missing edges may be reduced below a selected threshold value d.

FIG. 2 depicts a contact map prediction correction mechanism 200 in accordance with some embodiments of the present disclosure. The mechanism 200 illustrates how an example peptide (in particular, PDB ID: 1a2o) may be folded in accordance with the present disclosure. The contact map prediction correction mechanism 200 uses a PDB structure 212 to generate a PDB graph 222 and an MNNN structure 218 to generate an MNNN graph 228. In the mechanism 200 shown, both the PDB graph 222 and the MNNN graph 228 have a threshold value d of 6 Å.

The PDB graph 222 and the MNNN graph 228 are aggregated and compared to generate a missing edges graph 236. The missing edges in the missing edges graph 236 can be shrunk to render a corrected graph 246. The data from the missing edges graph 236 and the corrected graph 246 can be used to identify which edges an MNNN is likely to miss and, thus, compensate for the anticipated missing edges.

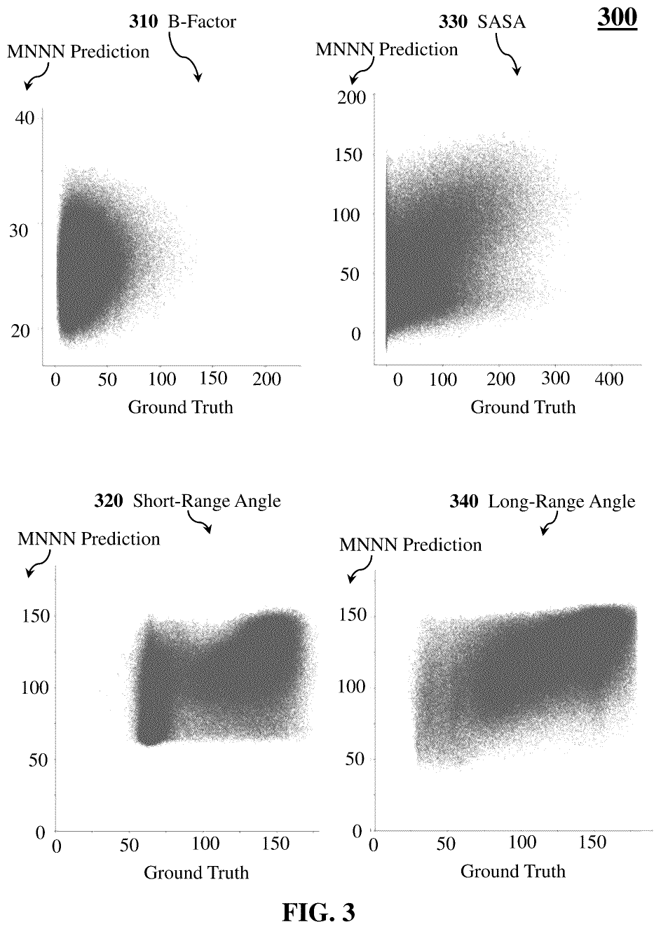

FIG. 3 illustrates a comparison graph set 300 in accordance with some embodiments of the present disclosure. The comparison graph set 300 includes a B-factor comparison graph 310, a SASA comparison graph 330, a short-range angle comparison graph 320, and a long-range angle comparison graph 340. Each graph in the comparison graph set 300 shows the MNNN prediction data plotted against the ground truth data. The comparison graph set 300 compares protein features predicted by the MNNN to the features obtained from the PDB files using a dataset of sequences of 8278 proteins.

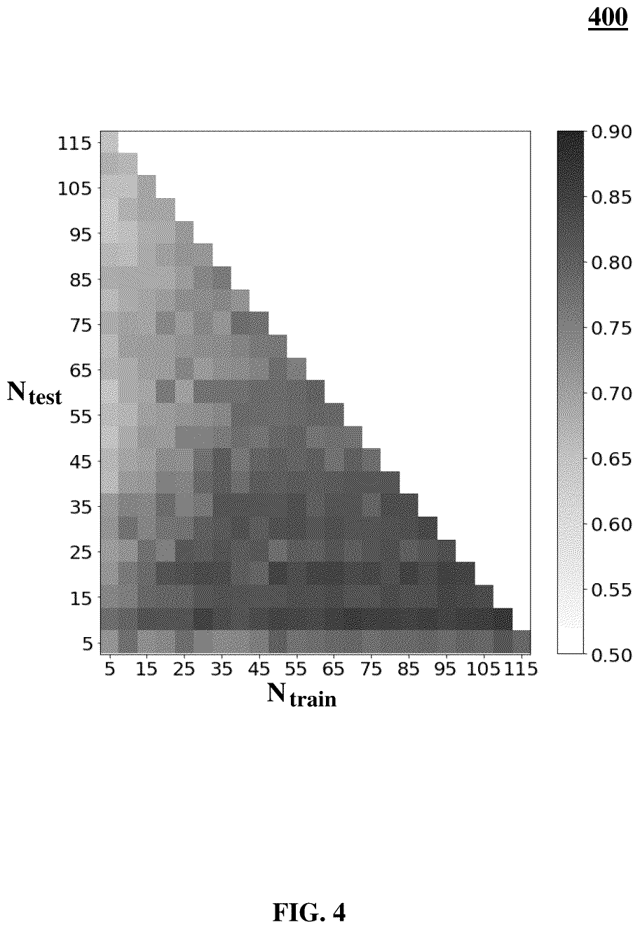

FIG. 4 illustrates a validation average precision score graph 400 in accordance with some embodiments of the present disclosure. The validation average precision score graph 400 shows the validation average precision (AP) score after 200 epochs as a function of Nₜᵣₐᵢₙ and Nₜₑₛₜ. The validation average precision score graph 400 compares the validation AP scores after training and testing the VGAE (e.g., VGAE 166 of FIG. 1) with different numbers of training and test graphs. In some embodiments, a subset of the dataset may be used to train and test the VGAE model. The validation average precision score graph 400 may compare the validation AP scores after training and testing the VGAE with different numbers of training and test graphs.

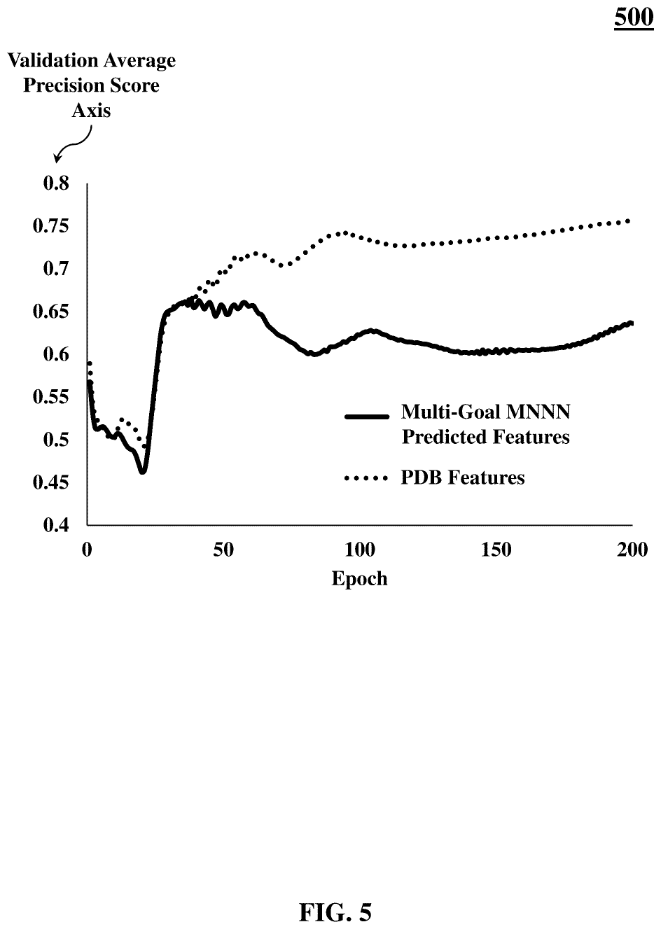

FIG. 5 depicts a learning curve graph 500 in accordance with some embodiments of the present disclosure. The parameters in Table 2 are adopted to generate the training and test graphs.

TABLE 2
Parameters for Generation of the Training and Test Graphs

| Parameter | Value | Description |
|---|---|---|
| Nₜᵣₐᵢₙ | 100 | number of training protein graphs |
| Nₜₑₛₜ | 20 | number of test protein graphs |
| k | 512 | number of clusters to classify φ − ψ angles |
| D_{idx} | 11 | minimum difference between indices of two |
| | | residues to define edges |
| d (Å) | 6.0 | maximum distance between Cα atoms of two |
| | | residues to define edges |

The learning curve graph 500 tracks the validation AP score over epochs to show the improvement in the molecular structure prediction with the prediction correction. The learning curve graph 500 plots the learning curve of a model (e.g., VGAE 166 of FIG. 1) trained with protein graphs in which the node features are predicted by a multi-goal MNNN as compared to the node features obtained from the PDB.

A computer-implemented method in accordance with the present disclosure may include training a neural network and predicting structural feature sets with the neural network. The method may include producing predicted structures with the neural network using the structural feature sets, converting the predicted structures into predicted graphs with predicted edges, and comparing predicted graphs to training graphs and predicted edges to training edges to obtain a comparison. The method may include training a model with the comparison, constructing a graph with the neural network using a node feature set, and reducing missing edges in the graph with the model.

In some embodiments of the present disclosure, the neural network may be a multi-scale neighborhood-based neural network (MNNN). In some embodiments, the multi-scale neighborhood-based neural network may be a multi-goal multi-scale neighborhood-based neural network (multi-goal MNNN).

In some embodiments of the present disclosure, the model may be a variational graph auto-encoder (VGAE).

In some embodiments of the present disclosure, the test missing edges may be reduced below a threshold value.

In some embodiments of the present disclosure, the method may include inputting amino acid code into the neural network with the structural feature set; the amino acid code may be used to produce the predicted structures.

In some embodiments of the present disclosure, the method may include using a molecular simulation program to produce the predicted structures.

In some embodiments of the present disclosure, the method may include labeling differences between predicted graphs and training graphs and labeling differences between predicted edges and training edges.

In some embodiments of the present disclosure, the method may include predicting a plurality of features of the predicted structure selected from the group comprising dihedral angles, B-factor, solvent-accessible surface area, long-range angles, and short-range angles.

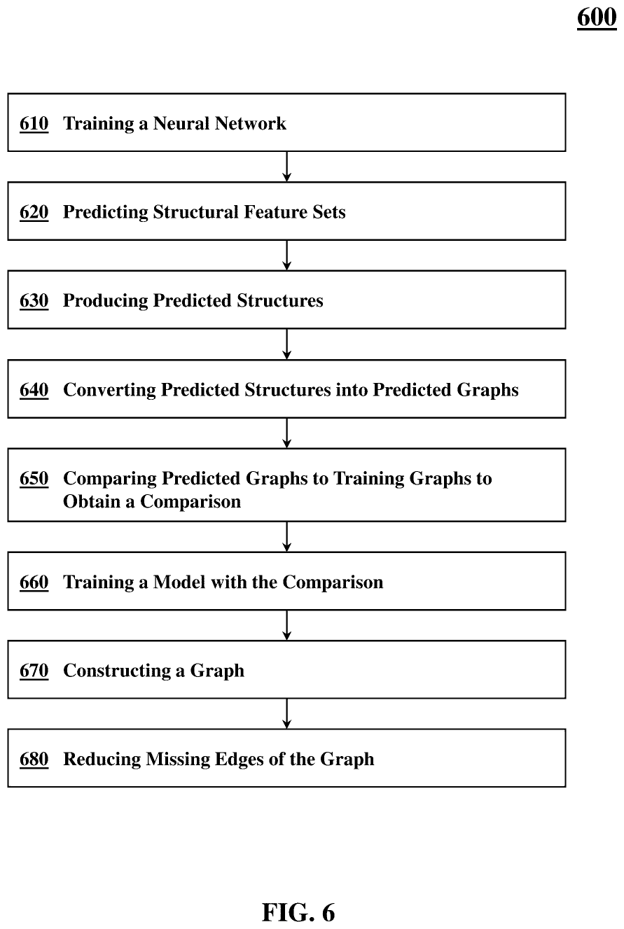

FIG. 6 depicts a structure prediction method 600 in accordance with some embodiments of the present disclosure. The method 600 may be used in system workflow (e.g., workflow 100 as shown in FIG. 1).

The structure prediction method 600 includes training 610 a neural network. In some embodiments, the neural network may be a MNNN such as a multi-goal MNNN. The method 600 includes predicting 620 structural feature sets with the neural network. Structural feature sets may include, for example, one or more features that describe a structure, such as a molecule's dihedral angles, B-factor, solvent-accessible surface area, long-range angles, and/or short-range angles.

The structure prediction method 600 includes producing 630 predicted structures. The predicted structures may be the structures of molecules such as peptide sequences and/or proteins. In some embodiments, a molecular simulation program (e.g., CHARMM) may be used to produce the predicted structures.

The structure prediction method 600 includes converting 640 the predicted structures into predicted graphs. The predicted graphs may have predicted edges. The method 600 includes comparing 650 the predicted graphs to training graphs to obtain a comparison between the prediction and the ground truth. The predicted edges of the predicted graphs may be compared to the training edges of the training graphs. In some embodiments, the differences between the predicted graphs and the training graphs may be labeled. In some embodiments, the differences between the predicted edges and the training edges may be labeled.

The structure prediction method 600 includes training 660 a model with the comparison. In some embodiments, the model may be a variational graph auto-encoder (VGAE). The structure prediction method 600 includes constructing 670 a graph with the trained neural network using a node feature set. The method 600 includes reducing 680 the missing edges of the graph with the trained model. In some embodiments, the missing edges may be reduced below a threshold value d.

A computer program product in accordance with the present disclosure may include a computer readable storage medium having program instructions embodied therewith. The program instructions may be executable by a processor to cause the processor to perform a function. The function may include training a neural network and predicting structural feature sets with the neural network. The function may include producing predicted structures with the neural network using the structural feature sets, converting the predicted structures into predicted graphs with predicted edges, and comparing predicted graphs to training graphs and predicted edges to training edges to obtain a comparison. The function may include training a model with the comparison, constructing a graph with the neural network using a node feature set, and reducing missing edges in the graph with the model.

In some embodiments of the present disclosure, the neural network may be a multi-scale neighborhood-based neural network (MNNN). In some embodiments, the multi-scale neighborhood-based neural network may be a multi-goal multi-scale neighborhood-based neural network (multi-goal MNNN).

In some embodiments of the present disclosure, the model may be a variational graph auto-encoder (VGAE).

In some embodiments of the present disclosure, the test missing edges may be reduced below a threshold value.

In some embodiments of the present disclosure, the function may include inputting amino acid code into the neural network with the structural feature set, and the amino acid code may be used to produce the predicted structures.

In some embodiments of the present disclosure, the function may include using a molecular simulation program to produce the predicted structures.

In some embodiments of the present disclosure, the function may include labeling differences between predicted graphs and training graphs and labeling differences between predicted edges and training edges.

In some embodiments of the present disclosure, the function may include predicting a plurality of features of the predicted structure selected from the group comprising dihedral angles, B-factor, solvent-accessible surface area, long-range angles, and short-range angles.

It is to be understood that although this disclosure includes a detailed description on cloud computing, implementation of the teachings recited herein are not limited to a cloud computing environment. Rather, embodiments of the present disclosure are capable of being implemented in conjunction with any other type of computing environment currently known or that which may be later developed.

Cloud computing is a model of service delivery for enabling convenient, on-demand network access to a shared pool of configurable computing resources (e.g., networks, network bandwidth, servers, processing, memory, storage, applications, virtual machines, and services) that can be rapidly provisioned and released with minimal management effort or interaction with a provider of the service. This cloud model may include at least five characteristics, at least three service models, and at least four deployment models.

Characteristics are as follows:

On-demand self-service: a cloud consumer can unilaterally provision computing capabilities, such as server time and network storage, as needed automatically without requiring human interaction with the service's provider.

Broad network access: capabilities are available over a network and accessed through standard mechanisms that promote use by heterogeneous thin or thick client platforms (e.g., mobile phones, laptops, and PDAs).

Resource pooling: the provider's computing resources are pooled to serve multiple consumers using a multi-tenant model with different physical and virtual resources dynamically assigned and reassigned according to demand. There is a sense of portion independence in that the consumer generally has no control or knowledge over the exact portion of the provided resources but may be able to specify portion at a higher level of abstraction (e.g., country, state, or datacenter).

Rapid elasticity: capabilities can be rapidly and elastically provisioned, in some cases automatically, to quickly scale out and rapidly release to quickly scale in. To the consumer, the capabilities available for provisioning often appear to be unlimited and can be purchased in any quantity at any time.

Measured service: cloud systems automatically control and optimize resource use by leveraging a metering capability at some level of abstraction appropriate to the type of service (e.g., storage, processing, bandwidth, and active user accounts). Resource usage can be monitored, controlled, and reported, providing transparency for both the provider and consumer of the utilized service.

Service models are as follows:

Software as a Service (SaaS): the capability provided to the consumer is to use the provider's applications running on a cloud infrastructure. The applications are accessible from various client devices through a thin client interface such as a web browser (e.g., web-based e-mail). The consumer does not manage or control the underlying cloud infrastructure including network, servers, operating systems, storage, or even individual application capabilities with the possible exception of limited user-specific application configuration settings.

Platform as a Service (PaaS): the capability provided to the consumer is to deploy onto the cloud infrastructure consumer-created or acquired applications created using programming languages and tools supported by the provider. The consumer does not manage or control the underlying cloud infrastructure including networks, servers, operating systems, or storage, but the consumer has control over the deployed applications and possibly application hosting environment configurations.

Infrastructure as a Service (IaaS): the capability provided to the consumer is to provision processing, storage, networks, and other fundamental computing resources where the consumer is able to deploy and run arbitrary software which may include operating systems and applications. The consumer does not manage or control the underlying cloud infrastructure but has control over operating systems, storage, and deployed applications, and the consumer possibly has limited control of select networking components (e.g., host firewalls).

Deployment models are as follows:

Private cloud: the cloud infrastructure is operated solely for an organization. It may be managed by the organization or a third party and may exist on-premises or off-premises.

Community cloud: the cloud infrastructure is shared by several organizations and supports a specific community that has shared concerns (e.g., mission, security requirements, policy, and/or compliance considerations). It may be managed by the organizations or a third party and may exist on-premises or off-premises.

Public cloud: the cloud infrastructure is made available to the general public or a large industry group and is owned by an organization selling cloud services.

Hybrid cloud: the cloud infrastructure is a composition of two or more clouds (private, community, or public) that remain unique entities but are bound together by standardized or proprietary technology that enables data and application portability (e.g., cloud bursting for load-balancing between clouds).

A cloud computing environment is service oriented with a focus on statelessness, low coupling, modularity, and semantic interoperability. At the heart of cloud computing is an infrastructure that includes a network of interconnected nodes.

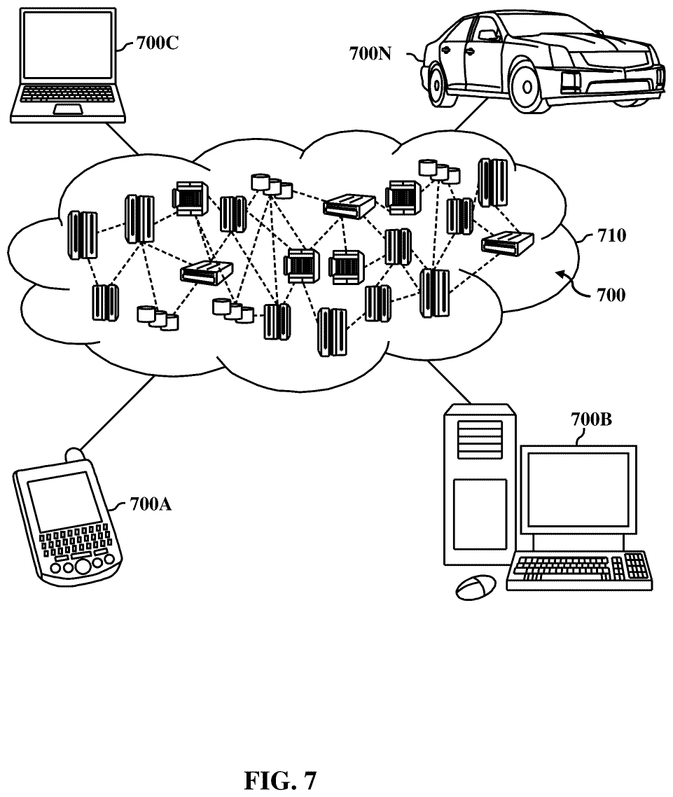

FIG. 7 illustrates a cloud computing environment 710 in accordance with embodiments of the present disclosure. As shown, cloud computing environment 710 includes one or more cloud computing nodes 700 with which local computing devices used by cloud consumers such as, for example, personal digital assistant (PDA) or cellular telephone 700A, desktop computer 700B, laptop computer 700C, and/or automobile computer system 700N may communicate. Nodes 700 may communicate with one another. They may be grouped (not shown) physically or virtually, in one or more networks, such as private, community, public, or hybrid clouds as described hereinabove, or a combination thereof.

This allows cloud computing environment 710 to offer infrastructure, platforms, and/or software as services for which a cloud consumer does not need to maintain resources on a local computing device. It is understood that the types of computing devices 700A-N shown in FIG. 7 are intended to be illustrative only and that computing nodes 700 and cloud computing environment 710 can communicate with any type of computerized device over any type of network and/or network addressable connection (e.g., using a web browser).

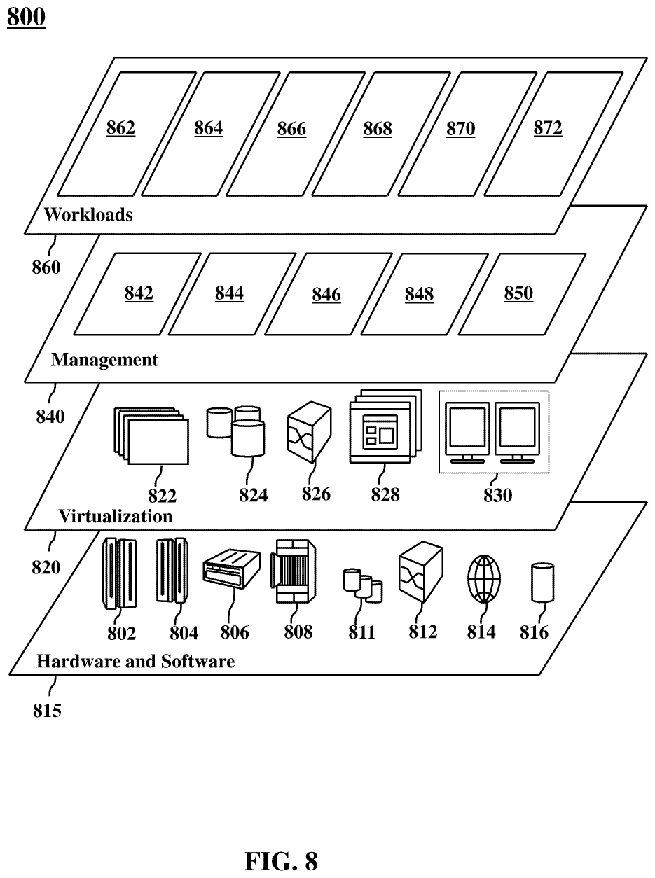

FIG. 8 illustrates abstraction model layers 800 provided by cloud computing environment 710 (FIG. 7) in accordance with embodiments of the present disclosure. It should be understood in advance that the components, layers, and functions shown in FIG. 8 are intended to be illustrative only and embodiments of the disclosure are not limited thereto. As depicted below, the following layers and corresponding functions are provided.

Hardware and software layer 815 includes hardware and software components. Examples of hardware components include: mainframes 802; RISC (Reduced Instruction Set Computer) architecture-based servers 804; servers 806; blade servers 808; storage devices 811; and networks and networking components 812. In some embodiments, software components include network application server software 814 and database software 816.

Virtualization layer 820 provides an abstraction layer from which the following examples of virtual entities may be provided: virtual servers 822; virtual storage 824; virtual networks 826, including virtual private networks; virtual applications and operating systems 828; and virtual clients 830.

In one example, management layer 840 may provide the functions described below. Resource provisioning 842 provides dynamic procurement of computing resources and other resources that are utilized to perform tasks within the cloud computing environment. Metering and pricing 844 provide cost tracking as resources and are utilized within the cloud computing environment as well as billing or invoicing for consumption of these resources. In one example, these resources may include application software licenses. Security provides identity verification for cloud consumers and tasks as well as protection for data and other resources. User portal 846 provides access to the cloud computing environment for consumers and system administrators. Service level management 848 provides cloud computing resource allocation and management such that required service levels are met. Service level agreement (SLA) planning and fulfillment 850 provide pre-arrangement for, and procurement of, cloud computing resources for which a future requirement is anticipated in accordance with an SLA.

Workloads layer 860 provides examples of functionality for which the cloud computing environment may be utilized. Examples of workloads and functions which may be provided from this layer include: mapping and navigation 862; software development and lifecycle management 864; virtual classroom education delivery 866; data analytics processing 868; transaction processing 870; and protein structure prediction using machine learning 872.

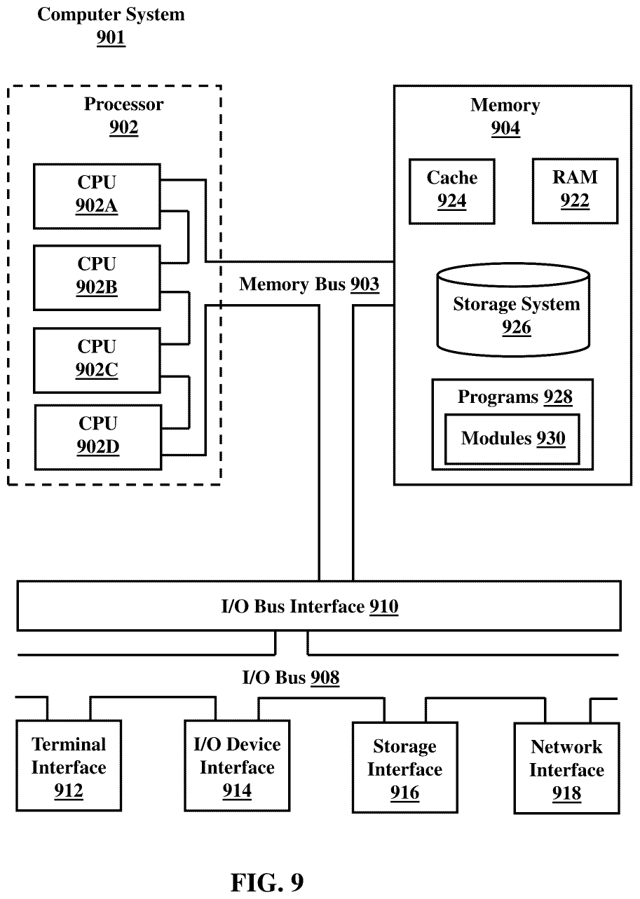

FIG. 9 illustrates a high-level block diagram of an example computer system 901 that may be used in implementing one or more of the methods, tools, and modules, and any related functions, described herein (e.g., using one or more processor circuits or computer processors of the computer) in accordance with embodiments of the present disclosure. In some embodiments, the major components of the computer system 901 may comprise a processor 902 with one or more central processing units (CPUs) 902A, 902B, 902C, and 902D, a memory subsystem 904, a terminal interface 912, a storage interface 916, an I/O (Input/Output) device interface 914, and a network interface 918, all of which may be communicatively coupled, directly or indirectly, for inter-component communication via a memory bus 903, an I/O bus 908, and an I/O bus interface unit 910.

The computer system 901 may contain one or more general-purpose programmable CPUs 902A, 902B, 902C, and 902D, herein generically referred to as the CPU 902. In some embodiments, the computer system 901 may contain multiple processors typical of a relatively large system; however, in other embodiments, the computer system 901 may alternatively be a single CPU system. Each CPU 902 may execute instructions stored in the memory subsystem 904 and may include one or more levels of on-board cache.

System memory 904 may include computer system readable media in the form of volatile memory, such as random access memory (RAM) 922 or cache memory 924. Computer system 901 may further include other removable/non-removable, volatile/non-volatile computer system storage media. By way of example only, storage system 926 can be provided for reading from and writing to a non-removable, non-volatile magnetic media, such as a “hard drive.” Although not shown, a magnetic disk drive for reading from and writing to a removable, non-volatile magnetic disk (e.g., a “floppy disk”), or an optical disk drive for reading from or writing to a removable, non-volatile optical disc such as a CD-ROM, DVD-ROM, or other optical media can be provided. In addition, memory 904 can include flash memory, e.g., a flash memory stick drive or a flash drive. Memory devices can be connected to memory bus 903 by one or more data media interfaces. The memory 904 may include at least one program product having a set (e.g., at least one) of program modules that are configured to carry out the functions of various embodiments.

One or more programs/utilities 928, each having at least one set of program modules 930, may be stored in memory 904. The programs/utilities 928 may include a hypervisor (also referred to as a virtual machine monitor), one or more operating systems, one or more application programs, other program modules, and program data. Each of the operating systems, one or more application programs, other program modules, and program data, or some combination thereof, may include an implementation of a networking environment. Programs 928 and/or program modules 930 generally perform the functions or methodologies of various embodiments.

Although the memory bus 903 is shown in FIG. 9 as a single bus structure providing a direct communication path among the CPUs 902, the memory subsystem 904, and the I/O bus interface 910, the memory bus 903 may, in some embodiments, include multiple different buses or communication paths, which may be arranged in any of various forms, such as point-to-point links in hierarchical, star, or web configurations, multiple hierarchical buses, parallel and redundant paths, or any other appropriate type of configuration. Furthermore, while the I/O bus interface 910 and the I/O bus 908 are shown as single respective units, the computer system 901 may, in some embodiments, contain multiple I/O bus interface units 910, multiple I/O buses 908, or both. Further, while multiple I/O interface units 910 are shown, which separate the I/O bus 908 from various communications paths running to the various I/O devices, in other embodiments some or all of the I/O devices may be connected directly to one or more system I/O buses 908.

In some embodiments, the computer system 901 may be a multi-user mainframe computer system, a single-user system, a server computer, or similar device that has little or no direct user interface but receives requests from other computer systems (clients). Further, in some embodiments, the computer system 901 may be implemented as a desktop computer, portable computer, laptop or notebook computer, tablet computer, pocket computer, telephone, smartphone, network switches or routers, or any other appropriate type of electronic device.

It is noted that FIG. 9 is intended to depict the representative major components of an exemplary computer system 901. In some embodiments, however, individual components may have greater or lesser complexity than as represented in FIG. 9, components other than or in addition to those shown in FIG. 9 may be present, and the number, type, and configuration of such components may vary.

The present disclosure may be a system, a method, and/or a computer program product at any possible technical detail level of integration. The computer program product may include a computer readable storage medium (or media) having computer readable program instructions thereon for causing a processor to carry out aspects of the present disclosure.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide, or other transmission media (e.g., light pulses passing through a fiber-optic cable) or electrical signals transmitted through a wire.

Computer readable program instructions described herein can be downloaded to respective computing/processing devices from a computer readable storage medium or to an external computer or external storage device via a network, for example, the Internet, a local area network, a wide area network, and/or a wireless network. The network may comprise copper transmission cables, optical transmission fibers, wireless transmission, routers, firewalls, switches, gateway computers, and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present disclosure may be assembler instructions, instruction-set-architecture (ISA) instructions, machine instructions, machine dependent instructions, microcode, firmware instructions, state-setting data, configuration data for integrated circuitry, or source code or object code written in any combination of one or more programming languages, including an object oriented programming language such as Smalltalk, C++, or the like, and procedural programming languages, such as the “C” programming language or similar programming languages. The computer readable program instructions may execute entirely on the user's computer, partly on the user's computer, as a stand-alone software package, partly on the user's computer and partly on a remote computer, or entirely on a remote computer or server. In the latter scenario, the remote computer may be connected to the user's computer through any type of network, including a local area network (LAN) or a wide area network (WAN) or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present disclosure.

Aspects of the present disclosure are described herein with reference to flowchart illustrations and/or block diagrams of methods, apparatus (systems), and computer program products according to embodiments of the disclosure. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus, or other device to produce a computer implemented process such that the instructions which execute on the computer, other programmable apparatus, or other device implement the functions/acts specified in the flowchart and/or block diagram block or blocks.

The flowchart and block diagrams in the figures illustrate the architecture, functionality, and operation of possible implementations of systems, methods, and computer program products according to various embodiments of the present disclosure. In this regard, each block in the flowchart or block diagrams may represent a module, segment, or portion of instructions which comprises one or more executable instructions for implementing the specified logical function(s). In some alternative implementations, the functions noted in the blocks may occur out of the order noted in the figures. For example, two blocks shown in succession may, in fact, be accomplished as one step, executed concurrently, substantially concurrently, in a partially or wholly temporally overlapping manner, or the blocks may sometimes be executed in the reverse order depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

Although the present disclosure has been described in terms of specific embodiments, it is anticipated that alterations and modifications thereof will become apparent to the skilled in the art. The descriptions of the various embodiments of the present disclosure have been presented for purposes of illustration but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application, or the technical improvement over technologies found in the marketplace or to enable others of ordinary skill in the art to understand the embodiments disclosed herein. Therefore, it is intended that the following claims be interpreted as covering all such alterations and modifications as fall within the true spirit and scope of the disclosure.

What is claimed is:

1. A system, said system comprising:
a memory; and
a processor in communication with said memory, said processor being configured to perform operations, said operations comprising:
training a neural network;
predicting structural feature sets with said neural network;
producing predicted structures with said neural network using said structural feature sets;
converting said predicted structures into predicted graphs with predicted edges;
comparing predicted graphs to training graphs and predicted edges to training edges to obtain a comparison;
training a model with said comparison;
constructing a graph with said neural network using a node feature set; and
reducing missing edges in said graph with said model.

2. The system of claim 1, wherein:
said neural network is a multi-scale neighborhood-based neural network.

3. The system of claim 1, wherein:
said model is a variational graph auto-encoder.

4. The system of claim 1, said operations further comprising:
inputting amino acid code into said neural network with said structural feature set, wherein said amino acid code is used to produce said predicted structures.

5. The system of claim 1, said operations further comprising:
using a molecular simulation program to produce said predicted structures.

6. The system of claim 1, said operations further comprising:
labeling differences between predicted graphs and training graphs; and
labeling differences between predicted edges and training edges.

7. The system of claim 1, said operations further comprising:
predicting a plurality of features of said predicted structure selected from the group consisting of dihedral angles, B-factor, solvent-accessible surface area, long-range angles, and short-range angles.

8. A computer-implemented method, said method comprising:
training a neural network;
predicting structural feature sets with said neural network;
producing predicted structures with said neural network using said structural feature sets;
converting said predicted structures into predicted graphs with predicted edges;
comparing predicted graphs to training graphs and predicted edges to training edges to obtain a comparison;
training a model with said comparison;
constructing a graph with said neural network using and a node feature set; and
reducing missing edges in said graph with said model.

9. The computer-implemented method of claim 8, wherein:
said neural network is a multi-scale neighborhood-based neural network.

10. The computer-implemented method of claim 9, wherein:
said multi-scale neighborhood-based neural network is a multi-goal multi-scale neighborhood-based neural network.

11. The computer-implemented method of claim 8, wherein:
said model is a variational graph auto-encoder.

12. The computer-implemented method of claim 8, further comprising:
inputting amino acid code into said neural network with said structural feature set, wherein said amino acid code is used to produce said predicted structures.

13. The computer-implemented method of claim 8, further comprising:
using a molecular simulation program to produce said predicted structures.

14. The computer-implemented method of claim 8, further comprising:
labeling differences between predicted graphs and training graphs; and
labeling differences between predicted edges and training edges.

15. The computer-implemented method of claim 8, further comprising:
predicting a plurality of features of said predicted structure selected from the group consisting of dihedral angles, B-factor, solvent-accessible surface area, long-range angles, and short-range angles.

16. A computer program product, said computer program product comprising a computer readable storage medium having program instructions embodied therewith, said program instructions executable by a processor to cause said processor to perform a function, said function comprising:
training a neural network;
predicting structural feature sets with said neural network;
producing predicted structures with said neural network using said structural feature sets;
converting said predicted structures into predicted graphs with predicted edges;
comparing predicted graphs to training graphs and predicted edges to training edges to obtain a comparison;
training a model with said comparison;
constructing a graph with said neural network using a node feature set; and
reducing missing edges in said graph with said model.

17. The computer program product of claim 16, wherein:
said neural network is a multi-scale neighborhood-based neural network.

18. The computer program product of claim 16, wherein:
said model is a variational graph auto-encoder.

19. The computer program product of claim 16, said function further comprising:
using a molecular simulation program to produce said predicted structures.

20. The computer program product of claim 16, said function further comprising:
predicting a plurality of features of said predicted structure selected from the group consisting of dihedral angles, B-factor, solvent-accessible surface area, long-range angles, and short-range angles.
